(19) United States
(12) Patent Application Publication
(10) Pub. No.: US 2007/0142772 A1
(43) Pub. Date: Jun. 21, 2007

(54) Dual-Layer Medical Balloon

(75) Inventors: Susheel Deshmukh, Santa Rosa, CA (US); Raymond Godaire, Auburn, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/609,012

(22) Filed: Dec. 11, 2006

Related U.S. Application Data
(60) Provisional application No. 60/751,255, filed on Dec. 16, 2005.

(51) Int. Cl. A61M 29/00; A61M 31/00; A61M 37/00

(52) U.S. Cl. 604/103.06

(57) ABSTRACT

A dual-layer dilatation balloon includes an inner layer that includes a polymer selected from the group consisting of a polyester, polyether, polyamide and copolymers thereof, and an outer layer that includes a polyamide. The dual-layer balloon optionally further includes a stent disposed on the balloon. The stent is optionally a drug-eluting stent. A process for forming a dual-layer dilatation balloon includes forming a dual-layer extrudate having an outer layer that includes a polyamide and an inner layer that includes a polymer selected from the group consisting of a polyester, polyether, polyamide and copolymers thereof. The process also includes forming the dual-layer balloon from the dual-layer extrudate in a balloon forming machine, wherein the balloon has a hoop strength of about 10,000 to about 60,000 p.s.i.

### CROSS-REFERENCE TO RELATED APPLICATIONS

This application claims the benefit of priority from U.S. Provisional Patent Application No. 60/751,255, which was filed on Dec. 16, 2005 and is currently pending, the entire content of which is hereby incorporated by reference.

### BACKGROUND OF THE INVENTION

1. Field of the Invention

The present invention relates to the field of balloon dilatation. Specifically, the present invention relates to balloons for dilatation applications and a process for manufacturing the balloons.

2. Related Art

Angioplasty balloons are currently produced by a combination of extrusion and stretch blow molding. The extrusion process is used to produce the balloon tubing, which essentially serves as a pre-form. This tubing is subsequently transferred to a stretch blow-molding machine capable of axially elongating the extruded tubing. U.S. Pat. No. 6,328,710 B1 to Wang et al. discloses such a process, in which a tubular preform is extruded and blown to form a balloon. U.S. Pat. No. 6,210,364 B1; U.S. Pat. No. 6,283,939 B1 and U.S. Pat. No. 5,500,180, all to Anderson et al., disclose a process of blow-molding a balloon, in which a polymeric extrudate can be stretched in both radial and axial directions.

The materials used in balloons for dilatation are primarily thermoplastics and thermoplastic elastomers such as polyesters and their block co-polymers, polyamides and their block co-polymers and polyurethane block co-polymers. U.S. Pat. No. 5,290,306 to Trotta et al. discloses balloons made from polyesterether and polyetheresteramide copolymers. U.S. Pat. No. 6,171,278 to Wang et al. discloses balloons made from polyether-polyamide copolymers. U.S. Pat. No. 6,210,364 B1; U.S. Pat. No. 6,283,939 B1 and U.S. Pat. No. 5,500,180, all to Anderson et al., disclose balloons made from block copolymers.

The unique conditions under which balloon dilatation is performed requires extremely thin-walled, high-strength balloons that are flexible and trackable enough to be maneuvered through tiny vessels. Balloons made from high strength polymers, while exhibiting high burst strengths, exhibit less flexibility and trackability than desired. The addition of plasticizer to the materials increases the softness and flexibility of the balloon. However, the use of plasticizer can limit the balloons applicability as a bio-compatible material. Balloons that exhibit high burst strengths that can be used in stent delivery, but also exhibit high flexibility and trackability are desired. New balloon materials are therefore needed to tailor the properties of the balloon and produce high-strength and highly flexible balloons for medical applications.

### SUMMARY OF THE INVENTION

In one embodiment, the present invention relates to a dual-layer dilatation balloon comprising an inner layer that includes a polymer selected from the group consisting of a polyester, polyether, polyamide and copolymers thereof, and an outer layer that includes a polyamide. The dual-layer balloon optionally further comprises a stent disposed on the balloon. The stent is optionally a drug-eluting stent.

In another embodiment, the present invention relates to a process for forming a dual-layer dilatation balloon. The process comprises forming a dual-layer extrudate having an outer layer including a polyamide and an inner layer including a polymer selected from the group consisting of a polyester, polyether, polyamide and copolymers thereof, and forming the dual-layer balloon from the dual-layer extrudate in a balloon forming machine, wherein the balloon has a hoop strength of about 10,000 to about 60,000 p.s.i.

In another embodiment, the present invention relates to a dual-layer dilatation balloon comprising an inner and outer layer, wherein said inner layer includes polyester-polyamide block copolymer, said outer layer includes a nylon polyamide, and said dual-layer balloon has a hoop strength of about 10,000 to about 60,000 p.s.i.

In another embodiment, the present invention relates to a balloon dilatation catheter, comprising a tubular elongated catheter shaft having proximal and distal portions, and a dual-layer dilatation balloon disposed on the shaft. The balloon includes an inner layer that includes a polymer selected from the group consisting of a polyester, polyether, polyamide and copolymers thereof, and an outer layer that includes a polyamide.

Optionally, the catheter includes a stent disposed on the balloon.

These and other embodiments, advantages and features will become readily apparent in view of the accompanying schematic drawings and the following detailed description of the invention.

### BRIEF DESCRIPTION OF THE DRAWINGS

Embodiments of the present invention will now be described, by way of example only, with reference to the accompanying schematic drawings in which corresponding reference symbols indicate corresponding parts, and in which:

FIG. 1 is a schematic side view of a balloon dilatation catheter according to an embodiment of the present invention;

FIG. 2 is a schematic detailed cross-sectional view of area A of FIG. 1;

FIG. 3 is a schematic side view of a balloon dilatation catheter according to another embodiment of the present invention;

FIG. 4 is a schematic drawing of a process for forming a dual-layer dilatation balloon according to an embodiment of the present invention; and

FIG. 5 is a detailed cross-sectional view of an embodiment of a mold for forming the dual-layer dilatation balloon of the present invention.

### DETAILED DESCRIPTION OF THE INVENTION

It is desirable to improve the flexibility and trackability of dilatation balloons while maintaining a high degree of strength in the balloon. Preferably, these improvements are made while limiting the use of plasticizers, which can migrate out of the balloon. Improved flexibility and trackability would allow a surgeon to maneuver the balloon, and alternatively, a balloon and stent, through very small diameter vasculature that may have a large degree of blockage or plaque build-up. The high degree of strength provides the surgeon with maximum flexibility to inflate the balloon, and alternatively to deliver a stent upon inflation, without bursting the balloon. In order to improve the flexibility of standard balloons without the use of plasticizers, or alternatively, with the limited use of plasticizers, a softer and more flexible material is co-extruded with a high-strength material to form a dual-layer balloon.

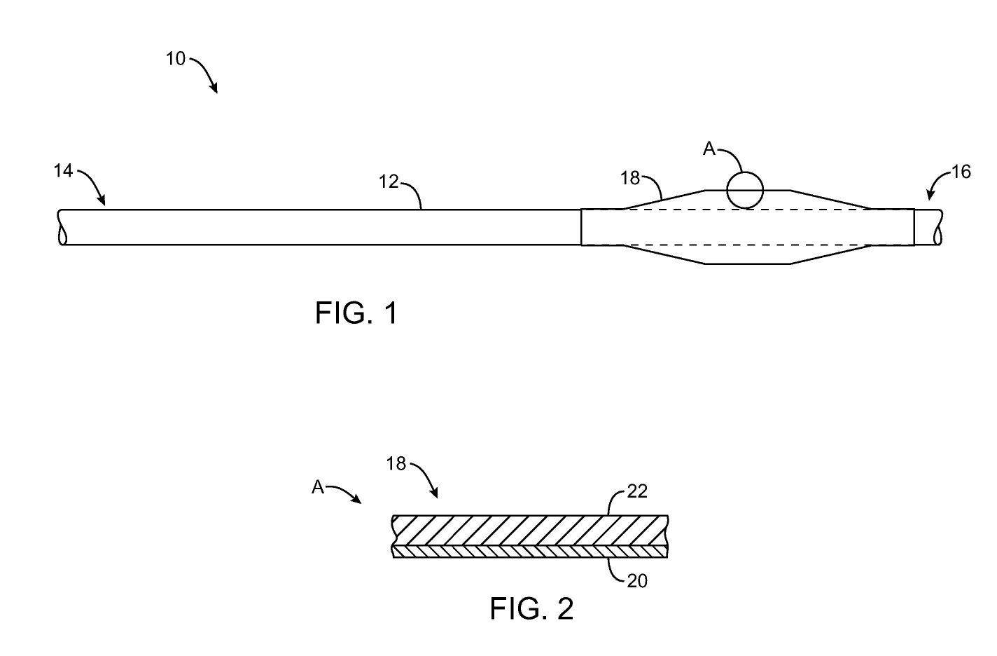

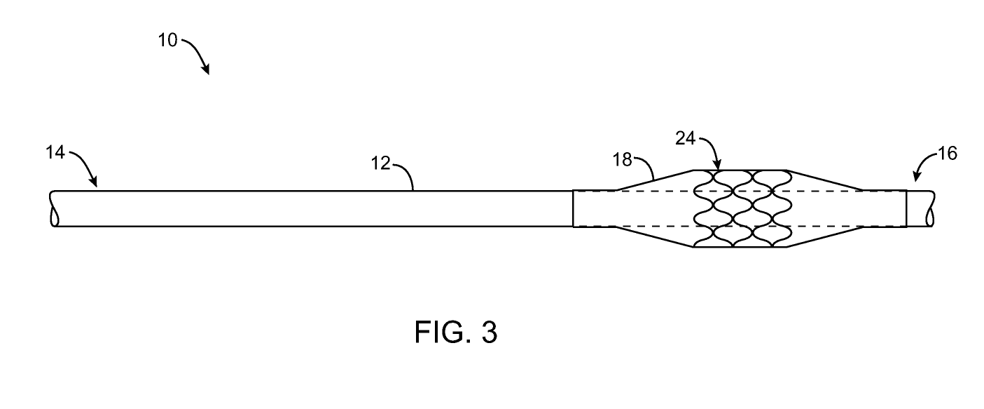

A balloon dilatation catheter 10 according to an embodiment of the invention is illustrated in FIG. 1. As illustrated, the catheter 10 includes a tubular elongated catheter shaft 12 having a proximal section 14 and a distal section 16, and a dual-layer dilatation balloon 18 connected to the distal section 16 of the shaft 12.

In one embodiment, the dual-layer dilatation balloon 18 includes an inner layer 20 that includes a polymer selected from the group consisting of a polyester, polyether, polyamide and copolymers thereof, and an outer layer 22 that includes a polyamide.

Dilatation is used herein to refer to the expandability of the balloon. Balloons of the present invention are expandable about 2% to about 40% greater than the original balloon size. Preferably, the expandability of the balloon is in the range of about 5% to about 20%.

Hoop strength is directly related to the maximum amount of pressure the balloon can withstand, for a given wall thickness, without failing or bursting. The balloons of the present invention have high hoop strengths for their given wall thickness. High hoop strength is used herein to refer to balloons having double wall thickness in the range of about 0.001 to about 0.05 inches for the dual-layer, and have hoop strengths greater than about 10,000 p.s.i. Balloons of the present invention preferably have hoop strengths of about 10,000 to about 60,000 p.s.i., alternatively, about 20,000 to about 50,000 p.s.i, alternatively, about 30,000 to about 40,000 p.s.i.

Polyamides for use in the outer layer 22 of balloons 18 of the present invention may include any polyamide that exhibits high hoop strength when formed into a dilatation balloon. Specific examples include, but are not limited to, nylon-type polyamides, such as, nylon-3, nylon-6, nylon-11, nylon-12, nylon-1/6, nylon-4/6, nylon-6/6 and nylon-6/10. A specific example includes, but is not limited to, AESNO® nylon-12, available from Atofina Chemicals, Inc. (Birsboro, Pa.). The molecular weight of the polyamide polymer used in the invention may be in the range of about 5,000 to about 5,000,000 Dalton. The type of polyamide used in any particular balloon depends on several factors, including, but not limited to, the type of polymer that will be co-extruded with the polyamide, and the desired final properties of the balloon. The dual-layer balloon 18 should have the same hoop strength or better than a balloon made from the outer layer polyamide alone, while having improved flexibility.

The inner layer 20 of the dual-layer balloon 18 according to embodiments of the present invention may comprise a polyester, polyether, polyamide or copolymers thereof. Any polyester, polyether, polyamide or copolymers thereof can be used as the inner layer 20, as long as the inner layer polymer is compatible with the polyamide outer layer 22 and the resulting dual-layer balloon 18 has high hoop strength and improved flexibility over a balloon made from only the outer layer polyamide. The molecular weight of the inner layer polymer used in the invention may be in the range of about 5,000 to about 5,000,000 Dalton. Specific examples of polymers for use as the inner layer include, but are not limited to, polyamide-polyether copolymers, such as block poly(ether-co-amide). Specific examples include, but are not limited to, PEBAX® copolymers, such as PEBAX® 6333 copolymer, available from Arkema, Inc. (Philadelphia, Pa.).

The dual-layer balloons 18 of the present invention optionally further comprise additives. Additives can be used in the inner layer 20, the outer polyamide layer 22 or in both layers. The term “additive” is used herein to refer to any material added to the polymer to affect the polymer's and/or the balloon's properties. Examples of additives for use in the invention include: plasticizers, fillers, antioxidants, colorants, crosslinking agents, impact strength modifiers, drugs and biologically active materials, such as compounds and molecules.

The dual-layer balloons 18 of the present invention optionally further comprise a plasticizer. The plasticizer may be used in the inner polymer layer 20, the outer polyamide layer 22 or in both layers. When the dual-layer balloon 18 is used for delivery of a drug-eluting stent, however, no plasticizer is preferably used in the outer polyamide layer.

The term “plasticizer” is used herein to mean any material that can decrease the flexural modulus of a polymer. The plasticizer may influence the morphology of the polymer and may affect the melting temperature and glass transition temperature. Examples of plasticizers include, but are not limited to: small organic and inorganic molecules, oligomers and small molecular weight polymers (those having molecular weight less than about 50,000), highly-branched polymers and dendrimers. Specific examples include: monomeric carbonamides and sulfonamides, phenolic compounds, cyclic ketones, mixtures of phenols and esters, sulfonated esters or amides, N-alkylarylsulfonamides, selected aliphatic diols, phosphite esters of alcohols, phthalate esters such as diethyl phthalate, dihexyl phthalate, dioctyl phthalate, didecyl phthalate, di(2-ethylhexy) phthalate and diisononyl phthalate; alcohols such as glycerol, ethylene glycol, diethylene glycol, triethylene glycol, oligomers of ethylene glycol; 2-ethylhexanol, isononyl alcohol and isodecyl alcohol, sorbitol and mannitol; ethers such as oligomers of polyethylene glycol, including PEG-500, PEG-1000 and PEG-2000; and amines such as triethanol amine.

The dual-layer balloons 18 of the present invention optionally further comprise a stent 24 disposed on the balloon 18. The dual-layer balloons 18 have high hoop strengths and allow for the delivery of the stent upon inflation of the balloon without bursting or puncturing the balloon. The stent 24 optionally comprises a drug or biologically active material. Any drug or biologically active material can be used in the stent. Specific examples include, but are not limited to, corticosteroids, such as dexamethasone, immunosuppresents, such as everolimus, sirolimus, and tacrolimus, and chemotherapeutic agents, such as paclitaxel. The drug or biologically active material elutes out of the stent and into the surrounding tissue over a controlled and predictable time. Preferably, no plasticizer is used in the outer layer 22 of the dual-layer balloon 18 when the balloon 18 is used for delivery of a drug-eluting stent.

In another embodiment of the present invention, the outer layer 22 of the dual layer balloon 18 includes a tough or relatively hard material, and the inner layer 20 includes a soft material. Having an outer layer that includes a tough material may impart high hoop strength and puncture resistance to the dual-layer balloon in stent delivery applications. Having an inner layer that includes a soft material may impart flexibility and trackability to the dual-layer balloon. In one example, tough materials for use as the outer layer include, but are limited to, those materials having a higher glass transition temperature than the soft materials used as an inner layer. In an alternative example, the outer layer includes a polyamide and the inner layer includes a polyester, polyether, polyamide or copolymers thereof.

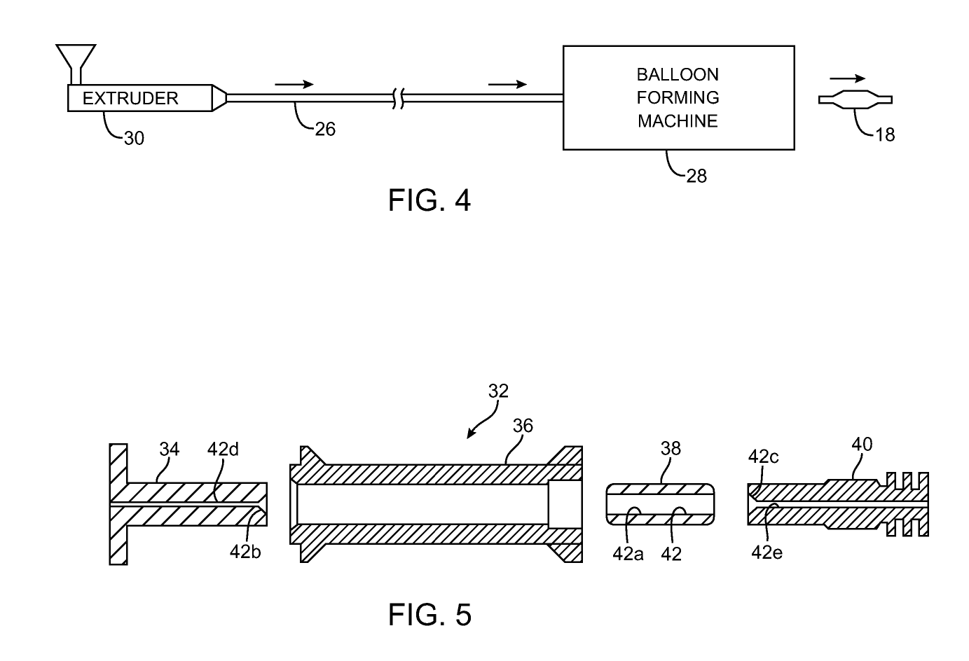

In another embodiment, the present invention relates to a process for forming a dual-layer dilatation balloon, which is schematically depicted in FIG. 4. The process comprises forming a dual-layer extrudate 26 comprising an outer layer including a polyamide and an inner layer including a polymer selected from the group consisting of a polyester, polyether, polyamide and copolymers thereof. The dual-layer balloon 18 is then formed from the dual-layer extrudate 26 in a balloon forming machine 28, such that the balloon has hoop strength of about 10,000 to about 60,000 p.s.i.

The dual-layer extrudate 26 may be formed in a tubular shape using an extruder 30. Extruders for use in the present invention include any extruder capable of forming dual-layer, tubular-shaped articles. Examples of extruders include, but are not limited to, single screw and double or twin screw extruders. In one embodiment, the material used for the outer layer polyamide and the inner layer polymer are loaded into different hoppers on the extruder in pellet or flake form. The outer layer polyamide and inner layer polymer are then extruded in different barrels, and co-extruded through a die, at which point, the two layers come together to form the dual-layer tubular extrudate 26. Preferably, no bonding layer is used and the dual-layer extrudate 26 is formed as a single article.

The extrusion temperature depends on the actual polymers being extruded. In general, the extrusion is performed at a temperature sufficient to melt the polyamide and inner layer polymers. For example, when extruding nylon 12, as the outer layer, and PEBAX® 6333 as the inner layer, the extruder may be heated such that the temperature of extrusion is about 220° C. to about 360° C., preferably about 260° C. to about 320° C. Tubular is used herein to mean a hollow, cylindrical-shaped article having an inner diameter, an inner circumference, an outer diameter and an outer circumference.

After forming the tubular extrudate 26, which may also be referred to as a parison or preform, the extrudate 26 is further processed in a balloon-forming step. The balloon-forming step is performed according to any one of the methods known to one of skill in the relevant art. For example, the stretching method of U.S. Pat. No. 5,948,345 to Patel et al., which is incorporated in its entirety herein by reference, can be used. According to the method of Patel et al., a length of tubing comprising a biaxially orientable polymer(s) or copolymer(s) is first provided having first and second portions with corresponding first and second outer diameters. Also provided is a mold 32 that defines an internal cavity having a generally cylindrical shape.

As shown in FIG. 5, the mold 32 comprises a first portion 34, a second portion 36, a third portion 38, and a fourth portion 40. The first portion 34, third portion 38, and fourth portion 40 are configured to be inserted into the second portion 36 in an abutting relationship so that the inner surfaces of the first portion 34, third portion 38, and fourth portion 40 define the balloon forming surface 42. The balloon forming surface 42 includes a central cylindrical portion 42a, defined by the third mold portion 38, and tapered portions 42b, 42c and neck portions 42d, 42e, defined by the first portion 34 and the fourth portion 40, as shown in FIG. 5. In an embodiment, the outer diameter of the extrudate 26 is larger than the diameter defined by the neck portion 42d of the first mold portion 34, and is smaller than the diameter of the neck portion 41 of the fourth portion 40, as well as the diameter of the central cylindrical portion 42a. The central cylindrical portion 42a may be sized relative to the outer diameter of the extrudate 26 so that the desired orientation and increase in hoop strength in the sidewall of the balloon 18 may be obtained.

To form the balloon 18, the extrudate 26 may be placed in the mold 32 and heated above the glass transition temperatures of the polymers in the two layers 20, 22. Pressure may then be applied to the extrudate 26 and the extrudate 26 may be longitudinally stretched such that it expands radially during the stretching. The extrudate 26 may be stretched about 4 to about 7 times the length of the tube's original length. In an embodiment, a pressure of about 300 to about 500 p.s.i. may be applied. A second higher pressure, about 15% to about 40% higher than the first pressure, may then be applied, and the resulting balloon 18 may be finally cooled below the glass transition temperatures of the polymers. One skilled in the relevant art appreciates that much of the stretching process can be performed by automated equipment in order to lower per unit costs. Upon completion of the stretching, the balloon 18 may be attached to the distal section 16 of the catheter shaft 12 by known methods to complete the production of the balloon dilation catheter 10.

After forming, the dual-layer balloon 18 of embodiments of the present invention may have a double wall thickness of about 0.001 inches to about 0.004 inches, and a diameter of about 2 to about 5 mm. In an embodiment, the inner layer 20 is about one quarter to about one third the thickness of the outer layer 22. In one example, the inner layer 20 has a (double wall) thickness of about 0.0004 inches and the outer layer 22 has a (double wall) thickness of about 0.0013 inches.

In another embodiment, the dual-layer balloon 18 may be made in accordance with the present invention having diameter of about 3.5 mm, a double wall thickness of about 0.0017 inches, and a burst strength of about 315 p.s.i. In an embodiment, the dual-layer balloon 18 may include PEBAX® 6333 as the inner layer 20, and nylon-12 as the outer layer 22.

In an experiment designed to evaluate the properties of balloons that were made in accordance with the present invention, three sets of balloons were made and properties of the balloons were measured. The average values of the balloon wall thicknesses, ratio of the balloon layers thicknesses, balloon burst strength, and balloon flexibility relative to the control are listed in Table I below. The control balloons were made from a single layer of nylon-12, and two types of dual layer balloons were also prepared in accordance with the present invention.

TABLE I
Comparison of Balloon Properties

| | Typical Balloon | | Typical Balloon | Typical Balloon | Typical Balloon |
|---|---|---|---|---|---|
| | Wall Thickness | Ratio of | Burst Strength | Flexibility | Flexibility |
| Balloon Type | (inches) | Balloon Layers | (psi) | (3 point bend) | (2D track) |
| Single Layer Control | 0.00068 | N/A | 336 | Control | Control |
| (Nylon-12) | | | | | |
| Dual Layer (Nylon-12 | 0.00076 | 75% Inner | 359 | 9% more flexible | 6% more flexible |
| Inner/PEBAX ® | | Layer/25% | | than control | than control |
| 6333 Outer) | | Outer Layer | | | |
| Dual Layer (PEBAX ® | 0.00075 | 25% Inner | 346 | 12% more flexible | 6% more flexible |
| 6333 Inner/ | | Layer/75% | | than control | than control |
| Nylon-12 Outer) | | Outer Layer | | | |

The balloon flexibility was measured by two separate flexibility tests, including a three point bend test, and a two dimensional trackability test, as would be appreciated by one of ordinary skill in the art. The balloons were subjected to the same testing conditions, so the results are presented as compared to the control. As indicated by the results listed in Table I, a more flexible balloon may be created by including a soft layer of PEBAX® 6333 in the balloon, without reducing the burst strength of the balloon. Even though the average thicknesses of the dual layer balloons were greater than the average thickness of the single layer control balloon, the dual layer balloons were more flexible than the single layer control balloon, on average. The properties listed in Table I are not intended to be limiting in any way and are merely provided as an example of embodiments of the present invention.

It will be understood by those skilled in the relevant art that various changes in form and details may be made therein without departing from the spirit and scope of the present invention as defined in the appended claims. Thus, the breadth and scope of the present invention should not be limited by any of the above-described exemplary embodiments, but should be defined only in accordance with the following claims and their equivalents.

What is claimed is:

1. A dual-layer dilatation balloon comprising an inner layer including a polymer selected from the group consisting of a polyester, polyether, polyamide and copolymers thereof, and an outer layer including a polyamide.

2. The balloon of claim 1, wherein said inner layer comprises a copolymer of a polyether and polyamide.

3. The balloon of claim 2, wherein said inner layer comprises block poly(ether-co-amide).

4. The balloon of claim 1, wherein said outer layer comprises a nylon polymer.

5. The balloon of claim 4, wherein said nylon polymer is nylon-3, nylon-6, nylon-11, nylon-12, nylon-1/6, nylon-4/6, nylon-6/6 or nylon-6/10.

6. The balloon of claim 5, wherein said nylon polymer is nylon 12.

7. The balloon of claim 1, wherein said balloon has a hoop strength of about 10,000 to about 60,000 p.s.i.

8. The balloon of claim 7, wherein said balloon has a hoop strength of about 20,000 to about 50,000 p.s.i.

9. The balloon of claim 1, wherein one or both of said inner and outer layers further comprise a plasticizer.

10. The balloon of claim 9, wherein said plasticizer is a carbonamide, sulfonamide, phenolic compound, cyclic ketone, mixture of phenols and esters, sulfonated ester, sulfonated amide, N-alkylarylsulfonamide, phthalate ester, amine, aliphatic diol or phosphite ester of an alcohol.

11. The balloon of claim 1, wherein one or both of said inner and outer layers further comprise at least one of a filler, antioxidant, colorant, crosslinking agent, impact strength modifier, drug or biologically active material.

12. The balloon of claim 1, further comprising a stent disposed on said balloon.

13. The balloon of claim 12, wherein said stent is a drug-eluting stent.

14. The balloon of claim 1, having a double wall thickness of about 0.001 to about 0.05 inches and a diameter of about 2 to about 5 mm.

15. The balloon of claim 14, wherein the wall thickness of said inner layer is about one quarter to about one third the thickness of said outer layer.

16. A balloon dilatation catheter, comprising:
a tubular elongated catheter shaft having proximal and distal portions; and
a dual-layer dilatation balloon disposed on said shaft, said balloon comprising an inner layer including a polymer selected from the group consisting of a polyester, polyether, polyamide and copolymers thereof, and an outer layer including a polyamide.

17. The catheter of claim 16, further comprising a stent disposed on said balloon.

18. The catheter of claim 17, wherein said inner layer comprises a copolymer of a polyether and polyamide.

19. The catheter of claim 18, wherein said inner layer comprises block poly(ether-co-amide).

20. The catheter of claim 16, wherein said outer layer comprises a nylon polymer.

21. The catheter of claim 20, wherein said nylon polymer is nylon-3, nylon-6, nylon-11, nylon-12, nylon-1/6, nylon-4/6, nylon-6/6 or nylon-6/10.

22. The catheter of claim 21, wherein said nylon polymer is nylon 12.

23. The catheter of claim 16, wherein said balloon has a hoop strength of about 10,000 to about 60,000 p.s.i.

24. The catheter of claim 23, wherein said balloon has a hoop strength of about 20,000 to about 50,000 p.s.i.

25. The catheter of claim 16, wherein said balloon has a double wall thickness of about 0.001 to about 0.05 inches and a diameter of about 2 to about 5 mm.

26. The catheter of claim 25, wherein the wall thickness of said inner layer is about one quarter to about one third the thickness of said outer layer.

27. A process for forming a dual-layer dilatation balloon, comprising:
forming a dual-layer extrudate having an outer layer including a polyamide and an inner layer including a polymer selected from the group consisting of a polyester, polyether, polyamide and copolymers thereof, and
forming said dual-layer balloon from said dual-layer extrudate in a balloon forming machine;
wherein said balloon has a hoop strength of about 10,000 to about 60,000 p.s.i.

28. The process of claim 27, wherein said extrudate forming step comprises co-extruding a polyamide and a second polymer selected from the group consisting of a polyester, polyether, polyamide and copolymers thereof.

29. The process of claim 27, wherein the thickness of said inner layer is about one quarter to about one third the thickness of said outer layer.

30. The process of claim 27, wherein said balloon has a hoop strength of about 20,000 to about 50,000 p.s.i.

31. A dual-layer dilatation balloon comprising an inner and outer layer, wherein said inner layer includes polyester-polyamide block copolymer, said outer layer includes a nylon polyamide, and said dual-layer balloon has a hoop strength of about 10,000 to about 60,000 p.s.i.

32. The balloon of claim 31, wherein said dual-layer balloon has a hoop strength of about 20,000 to about 50,000 p.s.i.

33. The balloon of claim 31, further comprising a stent disposed on said balloon.

34. The balloon of claim 31, wherein the wall thickness of said inner layer is about one quarter to about one third the thickness of said outer layer.
